(19) United States
(12) Patent Application Publication
(10) Pub. No.: US 2014/0200537 A1
(43) Pub. Date: Jul. 17, 2014

(54) Ostomy Ring

(71) Applicant: Lorena Jean Vogel, Albuquerque, NM (US)

(72) Inventor: Lorena Jean Vogel, Albuquerque, NM (US)

(21) Appl. No.: 13/744,279

(22) Filed: Jan. 17, 2013

(51) Int. Cl. A61F 5/44

(52) U.S. Cl. CPC A61F 5/4408
USPC 604/342

(57) ABSTRACT

The Ostomy Ring offers a reliable means of attaching an ostomy or other type bag to a patient via a surgically implanted magnetically susceptible material ring and an approximately sized magnetic ring placed over the skin and bag to secure the union and prevent leakage. Even a blind person could change their own bags without fear of leakage. A child could go to camp and ride a horse because there would be a reliable union between their stoma and their Ostomy Ring. In addition a semi-flexible Solid Ostomy Ring would also be available for the patient's use as an alternative to the outer magnetic ring. This would allow a person to go without their ostomy bag, and possibly be able to perform such essential duties such as going swimming, or going to their weddings, or going to their proms without as much chance of embarrassment.

### CROSS REFERENCE TO RELATED APPLICATIONS

This application is a continuation in part of application Ser. No. 12/802,278 filed Jun. 3, 2010 by inventor/applicant Lorena Jean Vogel.

### BACKGROUND OF THE INVENTION

1. Field of Invention

The present invention relates to a bio-medical device which attaches an ostomy bag to a person using a magnetic ring and a magnetically susceptible ring implanted under the skin.

2. Description of Prior Art

Adhesives, and the irregular contours of the human form make the problem of a reliable union between a stoma and an ostomy bag difficult, if not impossible, to perform. The conventional methods for making this uniontypically require using an adhesive that can cause irritation at the site, cause an imperfect union that can leak, and/or not allow any attachment at the site whatsoever.

U.S. Pat. No. 3,565,073 describes a “means attaching an appendage to an animal body comprising a stiff backing element embedded beneath the skin with a backing surface that faces outwardly, and an exteriorly carried complementing element held against the skin presenting a bearing surface that conforms to and is backed up by the backing surface of the embedded backing element.” This patent requires the use of “SpenceGell” which is no longer created by the Stryker Corporation of Michigan. In addition this patent grasps a specifically made bag on top of the outer ring while my patent could encorporate any manfactures ostomy appliance by holding it between the outer magnetic ring and the implanted ring.

U.S. Pat. No. 4,205,678 is an “ostomy bag securing apparatus, and method of applying same, characterized by implanting a plurality of small powerful permanent bar magnets through skin punctures to positions beneath the skin and surrounding a stoma, without surgical cutting of the skin, and applying a magnetically permeable collar, having a bag removable affixed thereto, on the outer surface of the skin, concentric with the magnets. Alternatively, the implanted bars may be magnetically permeable, but not permanently magnetized, and the permanently magnetized bars disposed on the collar which latter may be formed of non-magnetic material.” This procedure causes the patient to suffer not just one but nine procedures (the initial stoma procedure, and then the placement of eight bars around the stoma that can cause damage to the patient.) If the strong bar magnets are placed sub-dermally they may move and not retain the said shape of the ring and cause more pain for the patient and therefore more surgery to fix the issue. In addition with round magnets as shown in the art there would again be the problem of irregular contours that my patent is trying to alleviate.

U.S. Pat. No. 4,636,205 uses “a two part coupling for joining on ostomy bag to a pad or to a faceplate is characterized in that the two cooperating coupling parts are held together in a detachable manner by magnetic force.” The disparaging sizes seem to make this patent difficult to fabricate.

U.S. Pat. No. 7,422,578 is “a connecting ostomy device for coupling a collecting bag to a fixing device includes a first joining part connected to the fixing device and a second joining part connected to the collecting bag. The joining parts interact by adhesion in a plane practically perpendicular to the axis of orifices of the collecting bag of the fixing device in such a way that the transmission of the bag weight and the impermeability thereof are ensured by adhesion. The additional fixing elements ensure the alignment of the orifices of the fixing device and the collecting bag in a position thereof which is practically determined before the removal of the protective film from an adhesive surface. The connecting device used for ostomy.” There should be no adhesives used in the attachment of an ostomy bag.

### SUMMARY OF THE INVENTION

During the unfortunate time of ostomy-type surgery, a magnetically susceptible ring is implanted at the stoma site so that when a patient needs to replace their own ostomy bag they can do so by removing the magnetic ring placed over the ostomy bag and their skin (without any adhesive or any other layers involved.)

### BRIEF DESCRIPTION OF THE DRAWINGS

FIG. 1 shows a full view of a possible placement for The Ostomy Ring.

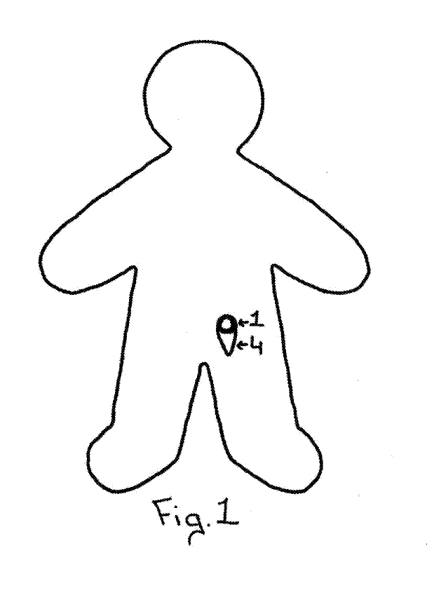

FIG. 2 shows a side view of The Ostomy Ring in place.

FIG. 3 shows a frontal view of The Ostomy Ring in place.

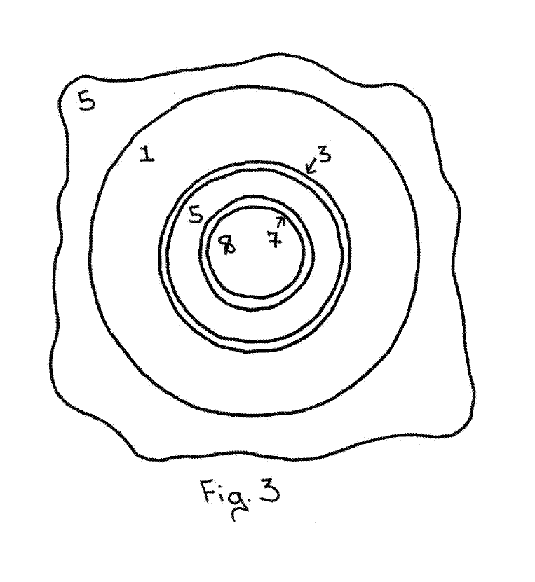

FIG. 4 shows a full view of a possible placement for The Solid Ostomy Ring.

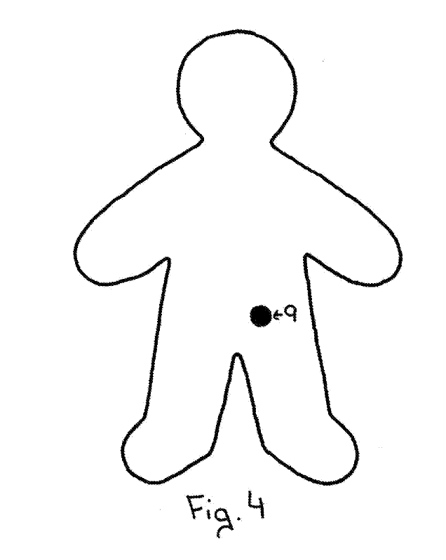

FIG. 5 shows a side view of the Solid Ostomy Ring in place.

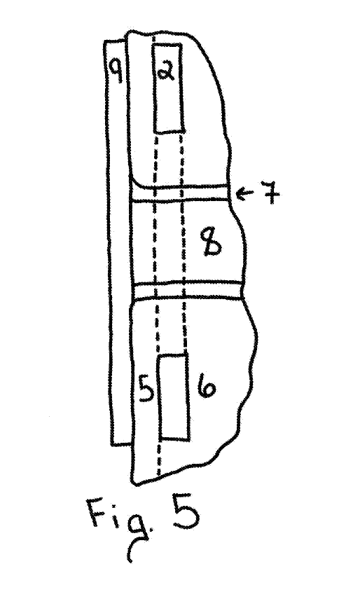

FIG. 6 shows a frontal view of The Solid Ostomy Ring in place.

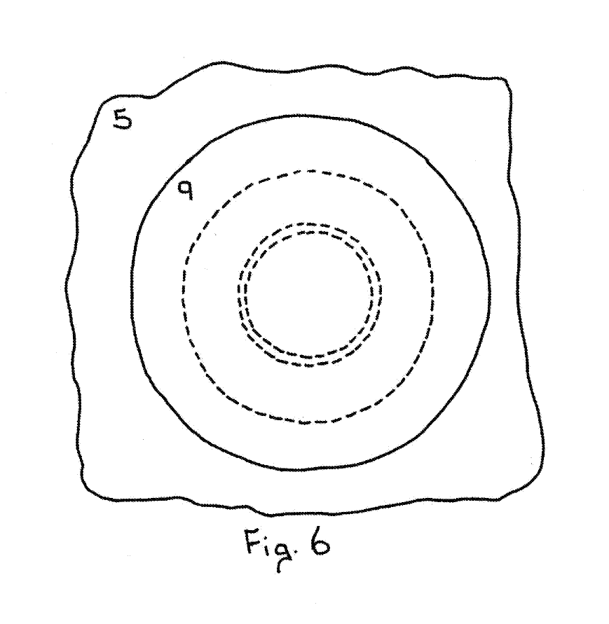

### DETAILED DESCRIPTION OF THE INVENTION

### 1. Definition of Reference Numerals in the Drawings

1. The Ostomy Ring
2. The magnetically susceptible ring that is implanted beneath the skin
3. The part of the ostomy bag held in place by The Ostomy Ring
4. The bag part of the ostomy bag
5. The epidermis
6. The dermis
7. The part of the intestine that wraps the stoma
8. The stoma
9. The Solid Ostomy Ring

### 2. How to Make the Invention

1. A magnetic ring of the same or slightly different size as 2.
2. A ring made of magnetically susceptible material (possibly covered by a polymeric material rated for medical applications.)
9. A solid ring/plate approximately the size of 1 made of a magnetic material.

A method for using an Ostomy Ring comprising the steps of: 1. forming a stoma, 2. implanting a magnetically susceptible ring possibly covered in a polymeric material rated for medical applications around the stoma and below the epidermis, 3. placing an Ostomy bag over the stoma and the epidermis, 4. placing the Ostomy Ring over the Ostomy bag causing the magnetic force between the magnetically susceptible ring and the Ostomy Ring to hold the Ostomy bag to the patient without the use of adhesives or other items.

An Ostomy Ring, comprised of a magnetic ring and a magnetically susceptible ring that is implanted beneath the skin, these are used to hold an Ostomy bag over a stoma between them using magnetism.

An Ostomy Ring, where the magnetically susceptible ring to be implanted under the skin might be covered in a polymeric material rated for medical applications.

An Ostomy Ring, where a magnetic ring is used on top of the implanted magnetically susceptible ring and the Ostomy bag to hold the bag in place.

An Ostomy Ring, where the magnetic ring and the magnetically susceptible ring are drawn together using magnetic force.

A method for using a Solid Ostomy Ring comprising the steps of: 1. forming a stoma, 2. implanting a magnetically susceptible ring possibly covered in a polymeric material rated for medical applications around the stoma and below the epidermis, 3. placing a Solid Ostomy Ring over the stoma and the epidermis. Placing the Solid Ostomy Ring over the epidermis seal for a short time the stoma will allow the patient to do almost anything without the use of adhesives or other items.

A Solid Ostomy Ring, comprised of a solid magnetic plate and a magnetically susceptible ring that is implanted beneath the skin, over a stoma.

A Solid Ostomy Ring, where a magnetically susceptible ring to be implanted under the skin that might be covered in a polymeric material rated for medical applications.

A Solid Ostomy Ring, where a solid magnetic plate could be placed directly over the stoma and on top of the magnetically susceptible ring without the need for an Ostomy bag for short periods of time.

A Solid Ostomy Ring, where the solid magnetic plate and the magnetically susceptible ring are drawn together using magnetic force.

### 3. Best Mode of the Invention

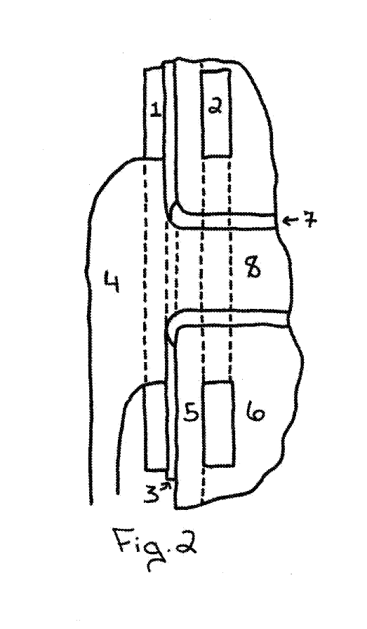

FIG. 2 shows a side sectional view of the best mode contemplated by the inventor of The Ostomy Ring according to the concepts of the present invention.

### 4. How to Use the Invention

The problems addressed by The Ostomy Ring are many as can be easily seen by those skilled in this art. The Ostomy Ring alleviates the need for any adhesive use when attaching an ostomy bag to a stoma. By doing so it also alleviates the trash associated with the need for adhesive use; i.e. boxes, tubes, films, etc. Using magnets to attach the ostomy bag is a green science using natural products and saving costs. Even blind patients and children would be able to change their own ostomy bag without having to go through extensive medical training to be able to do so.

### 5. Examples of the Invention

The Ostomy Ring will be appreciated by those skilled in the art that the present invention is not restricted to the particular preferred embodiments described with reference to the drawings, and that variations may be made therein without departing from the scope of the present invention as defined in the appended claims and equivalents thereof.

1. A method for using an Ostomy Ring comprising the steps of: forming a stoma, implanting a magnetically susceptible ring possibly covered in a polymeric material rated for medical applications around the stoma and below the epidermis, placing an Ostomy bag over the stoma and the epidermis, placing the Ostomy Ring over the Ostomy bag causing the magnetic force between the magnetically susceptible ring and the Ostomy Ring to hold the Ostomy bag to the patient without the use of adhesives or other items.

2. An Ostomy Ring comprising: a magnetic ring and a magnetically susceptible ring that is implanted beneath the skin , are used to hold an Ostomy bag over a stoma between them using magnetism.

3. An Ostomy Ring according to claim 2, wherein a magnetically susceptible ring to be implanted under the skin might be covered in a polymeric material rated for medical applications.

4. An Ostomy Ring according to claim 2, wherein a magnetic ring is used on top of the implanted magnetically susceptible ring and the Ostomy bag to hold the bag in place.

5. An Ostomy Ring according to claim 2, wherein the magnetic ring and the magnetically susceptible ring are drawn together using magnetic force.

6. A method for using a Solid Ostomy Ring comprising the steps of: forming a stoma, implanting a magnetically susceptible ring possibly covered in a polymeric material rated for medical applications around the stoma and below the epidermis, placing a Solid Ostomy Ring over the stoma and the epidermis, placing the Solid Ostomy Ring over the epidermis seal for a short time the stoma and allow the patient to do almost anything without the use of adhesives or other items.

7. A Solid Ostomy Ring according to claim 6, comprising: a solid magnetic plate and a magnetically susceptible ring that is implanted beneath the skin , over a stoma.

8. A Solid Ostomy Ring according to claim 6, wherein a magnetically susceptible ring to be implanted under the skin might be covered in a polymeric material rated for medical applications.

9. A Solid Ostomy Ring according to claim 6, wherein a solid magnetic plate could be placed directly over the stoma and on top of the magnetically susceptible ring without the need for an Ostomy bag for short periods of time.

10. A Solid Ostomy Ring according to claim 6, wherein the solid magnetic plate and the magnetically susceptible ring are drawn together using magnetic force.
